SEQUENCE LISTING (26 sequences)

SEQ ID NO: 1 - DNA, 20 bp, homo sapiens
accatgtgggtcccggttgt

SEQ ID NO: 2 - DNA, 21 bp, homo sapiens
gagttgataggggtgctcagg

SEQ ID NO: 3 - DNA, 28 bp, homo sapiens
acatcaaggaactcgatcgtatcattgc

SEQ ID NO: 4 - DNA, 20 bp, homo sapiens
ttgggcacttgcacagagat

SEQ ID NO: 5 - DNA, 20 bp, homo sapiens
ccaaggccttgcctgattgc

SEQ ID NO: 6 - DNA, 20 bp, homo sapiens
ttgggcacttgcacagagat

SEQ ID NO: 7 - DNA, 20 bp, homo sapiens
caccaagactgtgaggcaga

SEQ ID NO: 8 - DNA, 20 bp, homo sapiens
ccttgttcatgtaggcagca

SEQ ID NO: 9 - DNA, 21 bp, homo sapiens
cctcatcaagaaggatgtgga

SEQ ID NO: 10 - DNA, 20 bp, homo sapiens
caccacagatgtgtccgaga

SEQ ID NO: 11 - DNA, 20 bp, homo sapiens
gaccattgaggacctgagga

SEQ ID NO: 12 - DNA, 20 bp, homo sapiens
attgatgtcggcttccacac

SEQ ID NO: 13 - DNA, 20 bp, homo sapiens
ccagtctgtggagaacgaca

SEQ ID NO: 14 - DNA, 20 bp, homo sapiens
ctgagatttgggggcatcta

SEQ ID NO: 15 - DNA, 20 bp, homo sapiens
ccagtctgtggagaacgaca

SEQ ID NO: 16 - DNA, 20 bp, homo sapiens
atctgggcttgtaggccttt

SEQ ID NO: 17 - DNA, 26 bp, homo sapiens
ggcctgggagtctttgactccactac

SEQ ID NO: 18 - DNA, 26 bp, homo sapiens
atgtggagcccaaaccacagaaaatg

SEQ ID NO: 19 - DNA, 20 bp, homo sapiens
cgcggtgtcatggagaaact

SEQ ID NO: 20 - DNA, 20 bp, homo sapiens
cttcctgactggccaaatcc

SEQ ID NO: 21 - DNA, 23 bp, homo sapiens
ggtggtctcctctgacttcaaca

SEQ ID NO: 22 - DNA, 22 bp, homo sapiens
ttgctgtagccaaattcgttgt

SEQ ID NO: 23 - DNA, 20 bp, mus musculus
tgttcctacccccaatgtgt

SEQ ID NO: 24 - DNA, 20 bp, mus musculus
ggtcctcagtgtagcccaag

SEQ ID NO: 25 - DNA, 21 bp, homo sapiens
taggcgcgagctaagcaggag

SEQ ID NO: 26 - DNA, 25 bp, homo sapiens
gtaggcacactcaaacaacgactgg

(19) United States
(12) Patent Application Publication
(10) Pub. No.: US 2014/0304844 A1
(43) Pub. Date: Oct. 9, 2014

(54) XENOGRAFT MODEL OF HUMAN BONE METASTATIC PROSTATE CANCER

(71) Applicant: The Regents of the University of California, Oakland, CA (US)

(72) Inventors: Christina Jamieson, La Jolla, CA (US); Anna A. Kulidjian, La Jolla, CA (US); Catriona H. Jamieson, La Jolla, CA (US); Christopher J. Kane, San Diego, CA (US); Koichi Masuda, San Diego, CA (US); Christina CN Wu, Escondido, CA (US); Omer A. Raheem, San Diego, CA (US)

(21) Appl. No.: 14/351,491

(22) PCT Filed: Oct. 12, 2012
(86) PCT No.: PCT/US2012/060128

§ 371 Date: Apr. 11, 2014

Related U.S. Application Data
(60) Provisional application No. 61/546,996, filed on Oct. 13, 2011.

(51) Int. Cl. A01K 67/027; G01N 33/50

(52) U.S. Cl. CPC A01K 67/0271; G01N 33/5088
USPC 800/3; 800/10

(57) ABSTRACT

The disclosure herein provides a bone metastasis-derived prostate cancer xenograft model. The disclosure also provides methods for making a bone metastasis-derived prostate cancer xenograft model. In alternative embodiments, the disclosure provides compositions and methods for testing whether a drug, compound, diet, therapy or treatment is effective or efficacious for preventing, ameliorating, slowing the progress of, stopping or slowing the metastasis of, or for causing a full or partial remission of, a cancer, or a prostate cancer, or a human prostate cancer. The disclosure provides compositions and methods whether a drug, compound, diet, therapy or treatment is effective or efficacious for modifying or effecting the structure or organization or vascularization of a tumor microenvironment; or effects the growth, survival, phenotype or histology (tissue or organ structure or microenvironments) of connective tissue, bone cells, osteoblasts, osteocytes, osteoclasts, bone marrow cells, fibroblasts or angiogenic cells.

### CROSS REFERENCE TO RELATED APPLICATIONS

This application claims the benefit of priority to U.S. Provisional Application Ser. No. 61/546,996 entitled, “XENOGRAFT MODEL OF HUMAN BONE METASTATIC PROSTATE CANCER,” filed Oct. 13, 2011, which is incorporated by reference herein in its entirety, including all figures.

### FIELD OF THE INVENTION

The disclosure herein relates to oncology, cellular and developmental biology and drug discovery. In alternative embodiments, the disclosure herein provides a bone metastasis-derived prostate cancer xenograft model. In alternative embodiments, the disclosure provides methods for making a bone metastasis-derived prostate cancer xenograft model. In alternative embodiments, the disclosure provides compositions and methods for testing whether a drug, compound, diet, therapy or treatment is effective or efficacious for preventing, ameliorating, slowing the progress of, stopping or slowing the metastasis of, or for causing a full or partial remission of, a cancer, or a prostate cancer, or a human prostate cancer. In alternative embodiments, the disclosure provides compositions and methods whether a drug, compound, diet, therapy or treatment is effective or efficacious for modifying or effecting the structure or organization or vascularization of a tumor microenvironment; or effects the growth, survival, phenotype or histology (tissue or organ structure or microenvironments) of connective tissue, bone cells, osteoblasts, osteocytes, osteoclasts, bone marrow cells, fibroblasts or angiogenic cells.

### BACKGROUND OF THE DISCLOSURE

Bone metastases are detected in 80 to 100% of men who die of prostate cancer; such metastases lead to painfully debilitating fractures, spinal compression and rapid decline. In addition, prostate cancer bone metastases often become resistant to standard therapies including androgen deprivation, radiation and chemotherapy. A major limitation in understanding prostate cancer bone metastatic disease is that primary human prostate cancer bone metastasis tissues are rarely available for direct analysis and there are few models to elucidate mechanisms of interaction between the bone microenvironment and prostate cancer.

Research on prostate cancer bone metastasis has been limited because there are currently few models to elucidate mechanisms of interaction between the bone microenvironment and prostate cancer or in which to test therapies. Currently, there are three prostate cancer bone metastasis-derived orthotopic bone xenograft models: PC3, LAPC9 and VCaP. Xenograft transplantation of these cell lines into bone demonstrated the range of bone lesions produced by prostate cancer bone metastases: PC3 formed purely osteolytic lesions in intra-tibial xenografts, VCaP produced mixed osteoblastic/osteolytic lesions, while LAPC9 formed purely osteoblastic lesions.

### SUMMARY OF THE INVENTION

In alternative embodiments, the disclosure provides compositions and methods for making a bone metastasis-derived prostate cancer xenograft model, comprising:
(a) providing an immunodeficient non-human animal, or a non-human animal completely lacking B, T and NK cells,
wherein optionally the non-human animal is:
   a murine animal, or an animal of the subfamily Murinae or in the family Muridae,
   a rat or a mouse,
   an immunodeficient rat or mouse or animal of the subfamily Murinae or in the family Muridae, or a male rat or mouse or animal of the subfamily Murinae or in the family Muridae,
   an immunodeficient Rag2^{-/-};γ_{c}^{-/-} male rat or mouse or animal of the subfamily Murinae or in the family Muridae,
   a male non-human animal strain equivalent having the equivalent of a Rag2^{-/-};γ_{c}^{-/-} genotype and/or phenotype;
   an immunodeficient mouse or animal of the subfamily Murinae or in the family Muridae comprising (having contained therein) a human growth factor transgene or transgenes, wherein optionally the immunodeficient mouse is an NSG mouse, e.g., from the Jackson Laboratory, such as NOD.Cg-Prkdc^{scid} Il2rg^{tm1Wjl}/SzJ; or
   an immunodeficient murine or mouse strain or animal of the subfamily Murinae or in the family Muridae having a complete or partial human immune system reconstitution and/or modification;
(b) providing a mammalian or a human prostate cancer cell or cells,
wherein optionally the cell or cells are not passaged or cultured, or the cell or cells are cultured for one, two, three, four or five or more passages,

and optionally unpassaged or uncultured cells, or the passaged or cultured cells, are cryopreserved, lyophilized, freeze-dried or otherwise stored before placement (insertion) into the non-human animal,

and optionally the cells or cells are initially derived from a biopsy; and

(c) placing, injecting or inserting the human prostate cancer cell or cells into the non-human animal;

wherein optionally the cell or cells are injected, placed or inserted by injection subcutaneously (SQ), intravenously (IV), or directly into bone, or intra-femorally (IF), and/or intradermally.

In alternative embodiments, the non-human animal is murine animal or a mouse lacking all or part of a functional immune system or humoral immune system, and wherein the animal can be reconstituted fully or partly with a human immune system, or human humoral immune system (the ability to make human or human-like antibodies in the mouse), wherein optionally the murine animal is a XENOMOUSE™ (Abgenix Inc., Amgen, Fremont, Calif.), or a HuMAb-Mouse™ system (Medarex, Bristol Myers Squibb, Princeton, N.J.).

In alternative embodiments, the mammalian or human prostate cancer cell or cells are an advanced luminal prostate cancer bone metastatic cancer cell or cells, or have a prostate cancer phenotype or genotype or a human prostate cancer phenotype or genotype comprising one or more or all of the following: PSA⁺, AR⁺, K5⁻, K14⁻, K8⁺, K18⁺, AMACR⁺, N10(3.1⁺, and TMPRSS2:ERG⁻.

In alternative embodiments, the mammalian or human cell or cells, or the biopsy, is or are derived from a mixed osteoblastic and/or an osteolytic lesion.

In alternative embodiments, the invention provides bone metastasis-derived prostate cancer xenograft non-human animal or animal models made by a protocol or method comprising or consisting of a method disclosed herein.

In alternative embodiments, the disclosure provides compositions (e.g., the bone metastasis-derived prostate cancer xenograft non-human animal or animal models disclosed herein) and methods for testing whether a drug, compound, diet, therapy or treatment is effective or efficacious for: preventing, ameliorating, slowing the progress of, stopping or slowing the metastasis of, or for causing a full or partial remission of, a cancer, or a prostate cancer, or a human prostate cancer; or, modifies or effects the structure or organization or vascularization of a tumor microenvironment; or effects the growth, survival, phenotype or histology (tissue or organ structure or microenvironments) of connective tissue, bone cells, osteoblasts, osteocytes, osteoclasts, bone marrow cells, fibroblasts or angiogenic cells, comprising:

administering or applying the drug, compound, diet, therapy or treatment to the bone metastasis-derived prostate cancer xenograft model disclosed herein, or a bone metastasis-derived prostate cancer xenograft non-human animal or animal model made by a method comprising or consisting of a method disclosed herein, and

after administering or applying the drug, compound, diet, therapy or treatment to the bone metastasis-derived prostate cancer xenograft model, determining or measuring the effect of the drug, compound, diet, therapy or treatment on the cancer cells, or the effect of the drug, compound, diet, therapy or treatment on cells or composition or structure or organization or vascularization of a tumor microenvironment, or on connective tissue, bone cells, osteoblasts, osteocytes, osteoclasts, bone marrow cells, fibroblasts or angiogenic cells,

wherein optionally it is measured and/or determined whether the drug, compound, diet, therapy or treatment is effective or efficacious for preventing, ameliorating, slowing the progress of, stopping or slowing the metastasis of, or for causing a full or partial remission of, a cancer, or a prostate cancer, or a human prostate cancer,

and optionally it is measured and/or determined whether the drug, compound, diet, therapy or treatment is effective or efficacious for preventing, ameliorating, slowing the progress of, stopping or slowing a bone lesion formation, or effects bone or effects osteolysis, osteoschlerosis, osteoporosis, or osteopetrosis caused by a cancer, a prostate cancer or a human prostate cancer or cells,

and optionally a drug, compound, diet, therapy or treatment known to be effective or efficacious is co-administered, or administered to the same or similar animal, as a positive control.

In alternative embodiments, disclosed herein are immunocompromised mouse models in which PCSD1 cells are transplanted subcutaneously (SQ), intravenously (IV), or directly into bone, or intra-femorally (IF), and/or intradermally and a cancer forms. In an aspect of this embodiment, the cancer expresses AR, NKX3.1, Keratins 8 and 18 and AMACR. In other aspects of this embodiment, the PCSD1 cells are transplanted intrafemorally and the cancer that forms is bone cancer. In alternative embodiments, the bone cancer has osteolytic and osteoblastic lesions. In alternative embodiments, the transplanted cells are from a prostate metastatic bone cancer that is both osteolytic and osteoblastic and expresses AR, NKX3.1, Keratins 8 and 18 and AMACR. In alternative embodiments, the immunocompromised mouse is NOD.Cg-Prkdc^{scidIl2rgtm1Wjl}/SzJ. In still other aspects of this embodiment, the mouse model is used as a screening method of therapeutic agents for prostate cancer that has metastasized to bone, which comprises administering a test substance to the PCSD-1 mouse model or mouse model derived from transplanted cells from a prostate metastatic bone cancer that is both osteolytic and osteoblastic and expresses AR, NKX3.1, Keratins 8 and 18 and AMACR.

The details of one or more embodiments of the invention are set forth in the accompanying drawings and the description below. Other features, objects, and advantages of the disclosure herein will be apparent from the description and drawings, and from the claims.

All publications, patents, patent applications cited herein are hereby expressly incorporated by reference for all purposes.

### BRIEF DESCRIPTION OF THE DRAWINGS

FIG. 1. Intra-femoral transplantation of PCSD1 (Prostate Cancer San Diego 1) cells generated xenograft tumors in mice. Tumor cells isolated directly from a patient-derived femoral bone metastasis were transplanted intra-femorally into Rag2^{-/-};γ_{c}^{-/-} male mice. Tumor growth was observed in the tumor-injected (right) leg of intra-femorally transplanted mice but not in the un-injected, contra-lateral (left) leg as shown in three representative mice 10 weeks post-transplantation.

FIG. 2. PCSD1 xenograft tumors expressed human prostate PSA and human AR. (A) RNA was extracted from secondary transplant sub-cutaneous xenograft tumors (Passage 1, P1) and used for RT-PCR analysis of human prostate specific antigen (PSA) and human androgen receptor (AR). Human PSA and human AR-specific primers were used for PCR amplification of cDNA synthesized with reverse transcriptase (RT+) or without (RT−) and confirmed by sequencing of correctly sized bands. Human GAPDH-specific primers were used as an internal control. Human PSA and AR were expressed in the PCSD1 xenograft tumor and the human prostate cancer cell line, LAPC4, but not in the human chronic myelogenous leukemia (CML) cell line, K562. RNA from mouse spleen, bone marrow and liver did not express human PSA or AR. (B) Immunohistochemical analysis showed human PSA protein expression in PCSD1 xenograft tumors. Images show paraffin embedded (PPFE) sub-cutaneous PCSD1 secondary transplant xenograft sections stained with IgG isotype negative control or human PSA-specific antibody. (C) Upper panels show H and E stained intra-femoral PCSD1 secondary transplant xenograft cryosections at 10× and 20× magnification. Middle panels show cryosections from the left, un-injected, contralateral femurs immunostained with human PSA-specific antibody at 10× and 40×. Arrows show red blood cells in bone marrow. Lower panels show cryosections from secondary intra-femoral transplants of PCSD1 immunostained with anti-PSA.

FIG. 3. PCSD1 xenograft tumors express luminal-type epithelial, advanced prostate cancer biomarkers. RT-PCR analysis was performed on cDNA synthesized with reverse transcriptase (RT+) or without (RT−) from RNA purified from PCSD1 sub-cutaneous xenograft tumors, cultures of LAPC4, a human prostate cancer cell line and K562, a human CML cell line culture, as well as murine spleen, bone marrow and liver or H2O alone. Human specific primers for keratins 5, 8, 14 and 18, AMACR and NKX3.1, GAPDH and mouse-specific GAPDH were used to detect expression of these genes.

FIG. 4. MicroCT imaging of patient-derived intra-femoral (IF) PCSD1 xenografts revealed mixed osteolytic and osteoblastic bone lesions. MicroCT scanning was performed on femurs and tibia isolated from mice injected with primary patient-derived tumor in the right femur showed that areas of increased bone density and sclerosis were apparent in the femur in which the tumor was growing as shown above for mouse IF15. (A) P0 Bones osteolytic/osteoblastic MicroCT images; (B) 3D reconstruction of femur head.

FIG. 5. MicroCT imaging of PCSD1 secondary intra-femoral transplanted xenograft showing osteolytic at femur head and osteoblastic lesion formation along shaft of femur. (A). Osteoblastic MicroCT imaging of intra-femoral (IF) xenografts: CT and cross-sections. (B) Axial microCT scan series for comparison of femur cross-sections from un-injected, contra-lateral femur to PCSD1 tumor-injected femur.

### DETAILED DESCRIPTION OF THE EMBODIMENTS

In alternative embodiments, the disclosure herein provides a patient-derived model of bone metastatic prostate cancer. In alternative embodiments, the disclosure herein provides an animal model, e.g., a murine model, as a xenograft model of human bone metastatic prostate cancer, and methods for making and using these animals or models.

In other embodiments, the disclosure provides a bone metastasis-derived prostate cancer xenograft model. In alternative embodiments, the disclosure provides methods for making a bone metastasis-derived prostate cancer xenograft model. In alternative embodiments, described herein are compositions and methods for testing whether a drug, compound, drug combination, diet, therapy or treatment is effective or efficacious for preventing, ameliorating, slowing the progress of, stopping or slowing the metastasis of, or for causing a full or partial remission of, a cancer, or a prostate cancer, or a human prostate cancer. In alternative embodiments, described herein are compositions and methods to determine if a drug, compound, drug combination, diet, therapy or treatment is effective or efficacious for modifying or effecting the structure or organization or vascularization of a tumor microenvironment; or effects the growth, survival, phenotype or histology (tissue or organ structure or microenvironments) of connective tissue, bone cells, osteoblasts, osteocytes, osteoclasts, bone marrow cells, fibroblasts or angiogenic cells.

In still other embodiments, the disclosure provides methods of making these animals or models by injecting a specimen (e.g., an isolated, cultured and/or harvested cell or cells from a patient, or a biopsy sample from a patient) of patient-derived prostate cancer bone metastasis into the femurs of immunodeficient mice, wherein serially transplantable tumors were formed in the bone niche.

In alternative embodiments, described herein are animals or models (designated as the so-called “PCDS1 model”, where PCDS1 represents Prostate Cancer San Diego 1, as discussed in Example 1, below), and/or methods used to develop, validate or optimize new or improved compounds, treatments, diets or therapies, or to develop, validate or optimize new or improved test compounds, treatments, diets or therapies for the ability to prevent, reverse, ameliorate and/or inhibit prostate cancer growth in a bone-niche.

In alternative embodiments, described herein are animals or models (designated as a “PCDS 1 model”), and/or methods used to characterize, analyze and/or dissect the complex interactions between prostate cancer and the bone.

In still other embodiments disclosed herein are animals or models (designated as a “PCDS1 model”), and/or methods used to understand mechanisms of failure of standard-of-care therapy, or proposed new therapies for prostate metastatic bone cancer.

In alternative embodiments, described herein are animals or models (designated as a “PCDS 1 model”), and/or methods used to gain an understanding of the unexpected, discordant effects that are being reported for some new prostate (or other) cancer therapies.

In alternative embodiments, the disclosure herein includes use of a novel, patient-derived xenograft (PDX) model of bone metastatic prostate cancer (so called “PCDS1 model”), that is unique and, in alternative embodiments, can expand on and improve currently used therapies and treatments, e.g., in the following ways:
the so called “PCDS1 model” is a patient-derived, orthotopic xenograft model that closely reproduces human bone metastatic disease;
in alternative embodiments, in practicing the “PCDS1 model” low passage numbers of cancer cells can be used; cells can be cryopreserved from e.g., a primary tumor and/or subsequent passages; thus, the cells, and the model and methods disclosed herein become more representative and predictive of a bone metastatic prostate cancer in patients with these tumors;
in practicing the embodiments disclosed herein, methods of cancer cell or tumor preparation for serial transplantation are different from cell line models; for example, in one embodiment, a PCSD1 model was directly prepared from patient or xenograft tumor and not passaged in culture. Methods of cell dissociation and purification of PCSD1 cells were modified and optimized for the intra-femoral injection into the Rag2^{-/-};IL2Rgc^{-/-} mice.
in alternative embodiments, in practicing the “PCDS1 model” disclosed herein, transplantation into Rag2^{-/-};IL2Rgc^{-/-} immunodeficient mice which lack B, T and NK cells (vs. SCID mice which are not completely immunodeficient) is used.

As described in Example 1, below, PCSD1 xenografts tumors were characterized as advanced, luminal epithelial prostate cancer from a bone metastasis by RT-PCR and immunohistochemical biomarker analyses. MicroCT scanning showed that PCSD1 intra-femoral xenografts formed mixed osteoblastic/osteolytic lesions that closely resembled the bone lesions in the patient. Models disclosed herein are ready for use in pre-clinical drug testing as well as basic research, e.g., on castration-resistant prostate.

Bone metastasis models disclosed herein, including the so-called PCSD1 model, can be used to understand the complex mechanisms of interaction of prostate cancer with the bone microenvironment and the variation in response to therapies in different patients, types of bone lesions or stages of bone metastatic prostate cancer progression.

Current therapies such as bisphosphonates, radiation, anti-androgens, chemotherapy (docetaxel) often eventually fail in patients with castrate-resistant prostate cancer. In alternative embodiments, the so-called “PCSD1 model” disclosed herein is used not only to understand mechanisms of failure standard-of-care therapy but more importantly to develop new therapies alone or in combination with current therapies. In alternative embodiments, the so-called “PCSD1 model” can be used to gain an understanding of the unexpected, discordant effects of some new prostate cancer therapies that are being reported for bone metastatic prostate cancer. For example, in a Phase II Study of the new anti-androgen: abiraterone, it was found that approximately one third of patients with chemotherapy-naive metastatic castration-resistant prostate cancer displayed bone scan flare discordant with PSA serologic response. In other words, many patients with significantly lowered PSA levels after treatment with abiraterone still showed positive bone scans. Conversely, some patients treated with the new c-Met tyrosine kinase inhibitor cabozantinib (c-Met TKI, XL184) showed dramatic reductions in positive bone scans but, paradoxically, no decrease in their PSA levels. Accordingly, the bone metastasis models disclosed herein, including the so-called PCSD1 model, are effective for understanding the complex mechanisms of interaction of prostate cancer with the bone microenvironment and the variation in response to therapies in different patients, types of bone lesions or stages of bone metastatic prostate cancer progression. In alternative embodiments, the bone metastasis models disclosed herein, including the so-called PCSD1 model, comprise new primary prostate cancer bone metastasis-derived xenograft models to study metastatic diseases in the bone and to develop novel therapies for inhibiting prostate cancer growths in a bone-niche.

In other embodiments, the PCSD1 model when developed in sites other than bone, e.g., subcutaneously can be used to study and determine how tumors established at sites other than bone respond to different drugs and their characteristics as compared to the same tumor type in the bone environment.

### Kits and Instructions

The disclosure herein provides kits comprising compositions and/or instructions for practicing methods described herein. As such, kits, cells, vectors and the like can also be provided. In alternative embodiments, the disclosure herein provides kits comprising: a composition used to practice methods described herein, or a composition, a pharmaceutical composition or a formulation disclosed herein, and optionally comprising instructions for use thereof.

The embodiments disclosed herein will be further described with reference to the following examples; however, it is to be understood that the embodiments disclosed herein are not limited to such examples.

### EXAMPLES

### Example 1

### Prostate Cancer Bone Metastasis Model and Methods

This example provides data demonstrating that the methods and compositions described are, in alternative embodiments, effective as a prostate cancer bone metastasis model and methods. The so-called PCSD1 model disclosed herein is a validated patient-derived xenograft tumor model ready for use in pre-clinical drug testing as well as basic research on prostate cancer and tumor growth in the bone microenvironment/niche, e.g., a murine castration-resistant prostate cancer and tumor growth model in the bone.

Described herein is a patient-derived prostate cancer bone metastasis specimen that when injected into the femurs of immunodeficient mice formed serially transplantable tumors in the bone niche, the so-called “PCSD1 model”. The tumors were characterized by RT-PCR and immunohistochemistry as: PSA⁺, AR⁺, K5⁻, K14⁻, K8⁺, K18⁺, AMACR⁺, NKX3.1⁺, TMPRSS2;ERG⁻, advanced, luminal epithelial prostate cancer. MicroCT scanning showed that PCSD1 induced mixed osteoblastic/osteolytic lesions that closely recapitulated bone metastatic prostate cancer lesions in patients.

Described herein is the development and characterization of PCSD1 (Prostate Cancer San Diego 1), a novel patient-derived intra-femoral xenograft model of prostate bone metastatic cancer that recapitulates mixed osteolytic and osteoblastic lesions.

### Methods:

A femoral bone metastasis of prostate cancer was removed during hemiarthroplasty and transplanted into Rag2^{-/-};γ_{c}^{-/-} mice either intra-femorally or sub-cutaneously. Xenograft tumors that developed were analyzed for prostate cancer biomarker expression using RT-PCR and immunohistochemistry. Osteoblastic, osteolytic and mixed lesion formation was measured using micro-computed tomography (microCT).

### Results:

PCSD1 cells isolated directly from the patient formed tumors in all mice that were transplanted intra-femorally or sub-cutaneously into Rag2^{-/-};γ_{c}^{-/-} mice. Xenograft tumors expressed human prostate specific antigen (PSA) in RT-PCR and immunohistochemical analyses. PCSD1 tumors also expressed AR, NKX3.1, Keratins 8 and 18, and AMACR. Histologic and MicroCT analyses revealed that intra-femoral PCSD1 xenograft tumors formed mixed osteolytic and osteoblastic lesions. PCSD1 tumors have been serially passaged in mice as xenografts intra-femorally or sub-cutaneously as well as grown in culture.

### Conclusions:

PCSD1 xenografts tumors were characterized as advanced, luminal epithelial prostate cancer from a bone metastasis using RT-PCR and immunohistochemical biomarker analyses. PCSD1 intra-femoral xenografts formed mixed osteoblastic/osteolytic lesions that closely resembled the bone lesions in the patient. PCSD1 is a new primary prostate cancer bone metastasis-derived xenograft model to study metastatic disease in the bone and to develop novel therapies for inhibiting prostate cancer growth in the bone-niche.

### Methods

Tumor xenograft preparation: A primary prostate cancer bone metastasis sample was obtained from a lytic lesion in the proximal femur from a patient with castrate-resistant prostate cancer with mixed osteoblastic and osteolytic bone metastases and a Gleason score of 9 (5+4). Tumor specimen was prepared aseptically in biohazard safety cabinet according to standard protocols with minor modifications [28, 43-47]. Specimen was first minced with sterile razor blades to 1-3 mm³ sized pieces. Some of the minced tumor was snap frozen for genomic DNA and RNA extraction, cryopreserved in 10% DMSO/90% FBS, or fixed for immunohistochemistry. For sub-cutaneous transplantation the minced tumor was mixed 1:1 with High Concentration Matrigel (BD: Matrigel-Catalog Number: 354248). For intra-femoral injection, the minced tumor sample was disaggregated by digestion filtered through sterile, mesh filter (Falcon). Dissociated cells were centrifuged at 1200 RPM, 5 minutes, 4° C., washed three times and resuspended in Iscove's modified DMEM media, 10% FBS at 6.7×10⁶ cells/ml. Cells were mixed 1:1 with high concentration Matrigel for intra-femoral injection of 50,000 cells in 15 μl using UCSD animal welfare IACUC approved protocol. Remainder of the dissociated tumor cells were cryopreserved or used for DNA and RNA purification. All studies with human subjects were conducted with the approval of the University of California, San Diego School of Medicine Institutional Review Board. All patients provided written informed consent.

### Surgical Techniques:

Sub-cutaneous injections were performed with UCSD animal welfare IACUC approval using standard protocols [26, 28, 33]. Briefly, male Rag2^{-/-};γ_{c}^{-/-} mice 6-8 weeks old were anesthetized with ketamine/xylamine, skin sterilized with 70% ethanol, 2-3 mm incision with autoclaved dissection scissors, trochar (10 ml *LDEV-Free, 14-gauge catheter (SC injections) Terumo 14G IV Catheter), 100 μl of tumor/matrigel mix below skin right flank, skin flaps brought together and sealed with VetBond, mice revived post-surgery with Antisedan injected sub-cutaneously at base of neck ruff. For Intra-femoral injections mice were anesthesized by intra-peritoneal injection of a mix of 100 mg/kg ketamine and 10 mg/kg Xylazine and injections performed in a BL2 biosafety cabinet. Right hind limb was prepared under standard sterile conditions with 70% ethanol. Knee was held in flexed position and 25G needle (Monoject 200 25×5/8A) was used to make a port in the femoral plateau until there was no resistance that was used as a guide-hole for injection of 15 ul of the tumor cell/Matrigel suspension using a 0.3 ml syringe and 27G needle. Injection of sample was performed slowly with minimal resistance. Needle was withdrawn and leg immediately straightened, dabbed with antibiotic ointment (RX Neomycin, Polymyxin, Bacitracin Ophthalmic Ointment USP Sterile NDC 13985-017-55), on a sterile cotton tipped applicator (Q-Tips) and held for straight for approximately 1 minute. Mice were injected with Antisedan and placed on a warm Deltaphase Isothermal Pad, Ref 25-806 2 pc, Pur-Wraps, and carefully watched during recovery until ambulatory and active.

### Cells and Reagents:

Prostate cancer cell lines: LAPC4, was a gift from Dr. Lily Wu, UCLA, and VCaP, purchased from ATCC, were maintained in Iscove's media, 10% FBS, penicillin-streptomycin and K562, a chronic myelogenous leukemia cell line in 10% heat-inactivated FBS, RPMI, Pen-strep.

### RT-PCR:

Genomic DNA and RNA were extracted using mortar and pestle pulverization of flash frozen tumor pieces in liquid nitrogen and the Qiagen All-prep kit [47]. RNA was re-purified with RNeasy and treated with RNAse-free, DNase to remove contaminating genomic DNA. For cell lines and purified, dissociated xenograft tumor cells, RNA was extracted using Qiagen RNeasy mini-prep kit. cDNA synthesis was performed with Superscript III (Invitrogen, Inc.) according to manufacturer's protocol, and used for PCR (Taq polymerase, Monserate Biotechnology Group LLC, San Diego, Calif.). RT-PCR products were resolved on 1% agarose gels. All RT-PCR products of the correct size were verified by sequencing (Retrogen, Inc., San Diego, Calif.).

### Immunohistochemistry:

PSA immunostaining was carried out using rabbit anti-human PSA antibody (DAKO A0562) using standard protocols [55] performed by the Moores Cancer Center Histology Core, UCSD, La Jolla, Calif. Paraformaldehyde-fixed and paraffin embedded sections from sub-cutaneous PCSD1 xenografts were mounted and 6-μm sections were stained with H&E, anti-PSA, or rabbit IgG isotype control (DAKO N1699) using HRP goat anti-Rabbit as secondary antibody (Jackson 111-035-144) and AEC (Vector SK4200). For intra-femoral tumors, the tumor plus femur and tibia were dissected out as one, formalin-fixed and EDTA de-calcified according to Lavoie et al. [56]. Tissues were mounted in OCT, 6 μm cryosections were fixed in acetone, blocked in 1% BSA/PBS, incubated with anti-PSA or IgG isotype control antibody then processed as above.

### Micro CT Analyses:

Femurs of mice injected intra-femorally with PCSD1 were scanned by micro-computed tomography (μCT) SkyScan 1076 (Skyscan, Belgium) at the maximal potential 60 kV and 167 μA with 0.5 mm thick aluminum filter and at the voxel resolution of 9 μm. The μCT scans were performed over 360° of total rotation with each angular rotation step of 0.7°. The reconstructions, performed using the NRecon™ software package (Skyscan), are based on the Feldkamp algorithm and resulted in axial glayscale images. The 2D images were created using CTAn software package (Skyscan) [57]. The 3D μCT models of each femur were created using a 3D reconstruction software package (Mimics 14.0, Materialise, Belgium) [57, 58].

### Results

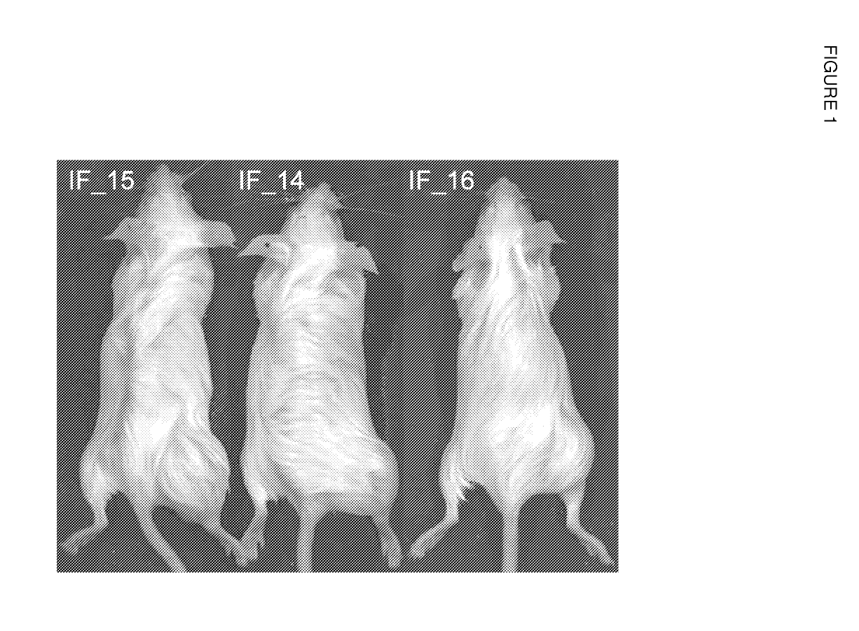

Patient derived-prostate cancer bone metastasis tumor specimen generated tumors in immunodeficient mice: Prostate tumor specimen was obtained from a castrate-resistant patient-derived femoral bone prostate metastasis and transplanted sub-cutaneously or intra-femorally into immunodeficient, male Rag2^{-/-};γ_{c}^{-/-} mice [59]. Minced tumor sample that was injected sub-cutaneously (SQ) produced xenograft tumors in all ten male Rag2^{-/-};γ_{c}^{-/-} mice. Disaggregated primary tumor cells that were injected intra-femorally (IF) generated tumors in all eight Rag2^{-/-};γ_{c}^{-/-} mice. As shown in FIG. 1, tumors were evident in three representative mice at ten weeks in the tumor-injected (right) leg of all intra-femorally transplanted mice but not in the un-injected, contra-lateral (left) leg. Therefore, the take-rate of the primary tumor sample was 100% in both the sub-cutaneous and intra-femoral niches. Tumors harvested from both sub-cutaneous and intra-femoral tumors have been serially transplanted at least three times both sub-cutaneously and intra-femorally thus far: P0 (primagraft), P1 and P2. Low passage PCSD1 tumors were cryopreserved and serially passaged as intra-femoral and sub-cutaneous xenografts. Tumor take-rates are shown in Table-2. The lower take-rate in the intra-femorally injected mice is most likely due to the significantly fewer tumor cells injected into the femur than sub-cutaneously. Approximately 5,000 tumor cells were injected per femur which was ˜10% of the total mixture of cells injected IF compared to minced tumor pieces ˜1 mm³ that were implanted sub-cutaneously. Mice injected IF with fewer as well as greater than 5,000 PCSD1 cells are currently being analyzed. Freshly harvested xenograft tumor cells as well as cryopreserved xenograft tumor cells have been used for long term in vitro culture experiments for testing novel compounds.

### PCSD1 Sub-Cutaneous and Intra-Femoral Xenograft Tumors Express PSA and AR:

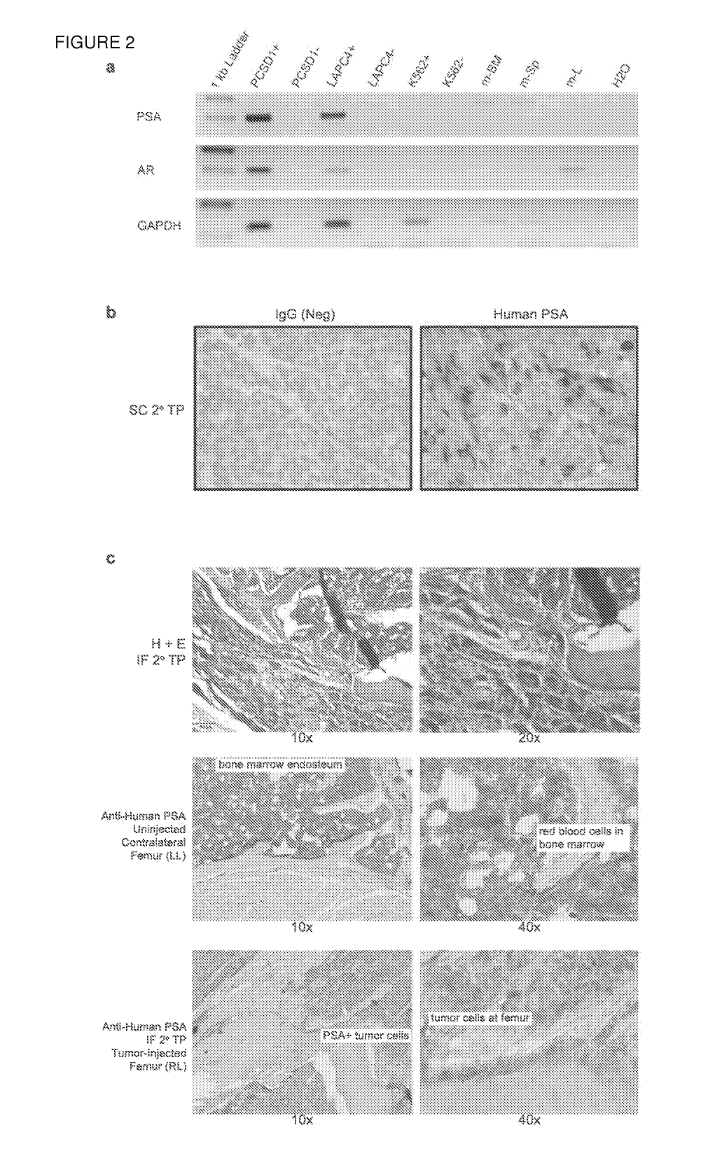

To demonstrate whether the xenograft tumors originated from prostate cancer in the patient bone metastasis specimen, the expression of prostate specific antigen (PSA) was measured. As shown in FIG. 2A, RT-PCR analysis showed the expression of human PSA in a sub-cutaneous PCSD1 xenograft tumor (P1) as well as in the human prostate cancer cell line, LAPC4, but not the human chronic myelogenous leukemia (CML) cell line, K562, nor murine bone marrow, spleen or liver [59]. Using primers for the full-length isoform of androgen receptor (AR) for RT-PCR demonstrated human androgen receptor (AR) expression in PCSD1 and LAPC4 (FIG. 2A). Therefore, PCSD1 xenograft tumors originated from human prostate cancer cells in the femoral bone metastasis.

PSA protein expression was determined using immunohistochemical staining of PCSD1 xenograft tumor sections. Cytoplasmic PSA staining was detected in cells in sub-cutaneous PCSD1 xenografts (FIG. 2B) and in intra-femoral xenografts from the right leg (FIG. 2C, IF 2° TP (RL), lower panels). Cytoplasmic PSA staining was not observed in femoral sections from the un-injected, contra-lateral left leg (LL). Red blood cells in the bone marrow space that showed up as slightly reddish brown in color that was not due to PSA immunostaining were seen in the un-injected intra-femoral sections (FIG. 2C middle panels). In the femur of the right leg, PCSD1 tumor cells were observed both in the endosteal bone marrow space where they were injected and having invaded extra-cortically surrounding the femur. Regions of osteolysis were observed in the immunostained sections and in H&E stained sections (FIG. 2C, upper panels) through which the tumor may have invaded and migrated outside of the bone.

### PCSD1 Xenograft Tumors Express Luminal Prostate Biomarkers:

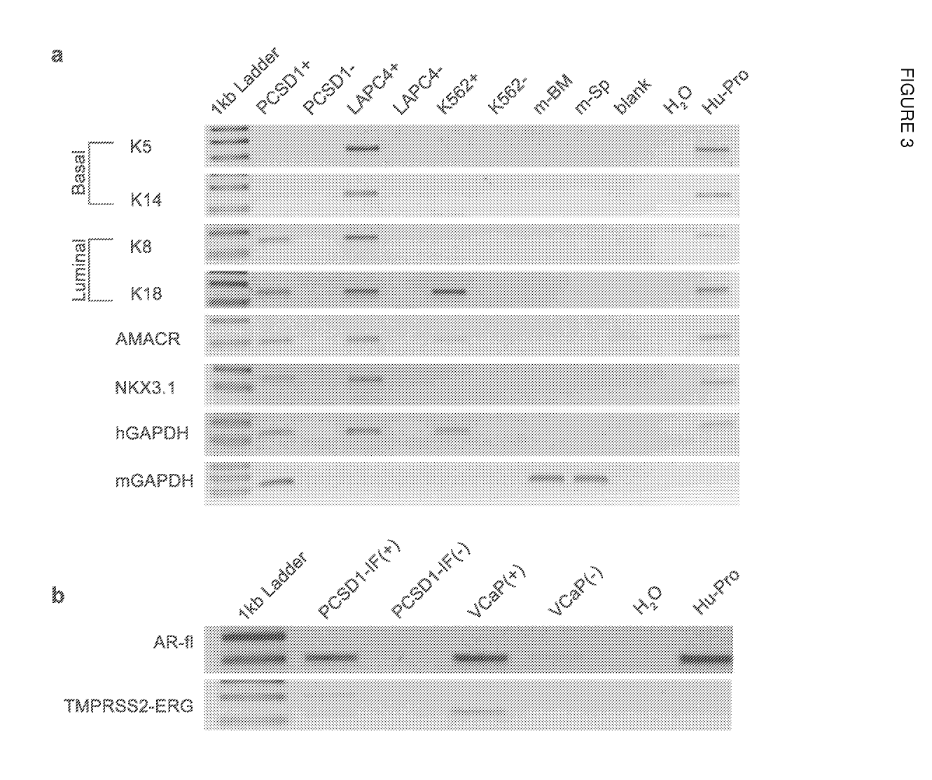

Further molecular analysis to characterize PCSD1 tumors was performed using RT-PCR on additional human prostate biomarkers. Expression of keratins 5 (K5) and 14 (K14) are characteristic of basal prostate epithelial cells whereas keratins 8 (K8) and 18 (K18) are expressed in luminal prostate epithelial cells [44-46, 50]. PCSD1 xenograft tumors expressed human K8 and K18 and very low levels of K5 and K14 (FIG. 3A). Interestingly, LAPC4 expressed all four of the keratins. PCSD1 and LAPC4 both expressed the prostate transcription factor NKX3.1 [46]. PCSD1 and LAPC4 also expressed AMACR, a biomarker that is often up-regulated in advanced prostate cancer [51]. The human specific GAPDH and mouse specific GAPDH were expressed in the PCSD1 xenografts indicating the presence of both human and murine cells within the xenograft tumor. Only human specific GAPDH was detected in the human cell lines LAPC4 and K562 that were grown in culture as expected. Correspondingly, the murine bone marrow and spleen tissues only expressed the mouse GAPDH. Taken together the results of the molecular analysis showed that PCSD1 is a luminal epithelial-type advanced prostate cancer [43-47, 50].

The TMPRSS2-ERG fusion gene is a frequent genomic rearrangement in prostate cancers that results in placing the ERG ETS-family transcription factor under the androgen-regulated expression of the TMPRSS2 gene [54]. RT-PCR was performed to determine whether this gene fusion event was present in PCSD1. While the fusion transcript was detected in VCaP cells as shown previously [54], the TMPRSS2-ERG gene fusion was not detected in PCSD1 (FIG. 3B). In addition, analysis of known alternative splicing variants of AR did not detect these in PCSD1 xenografts.

### PCSD1 Intra-Femoral Xenograft Forms Mixed Osteolytic and Osteoblastic Bone Lesions:

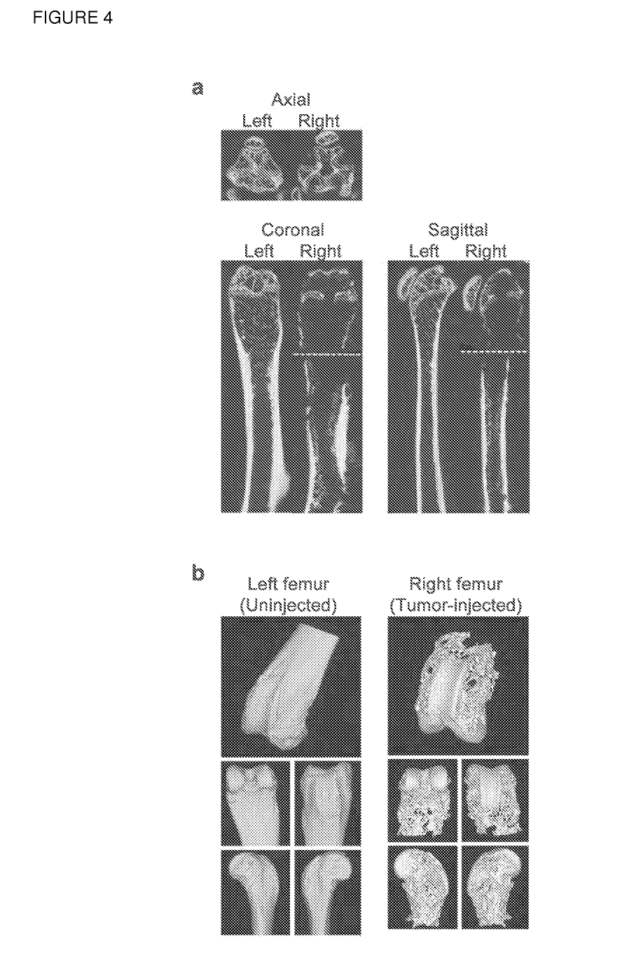

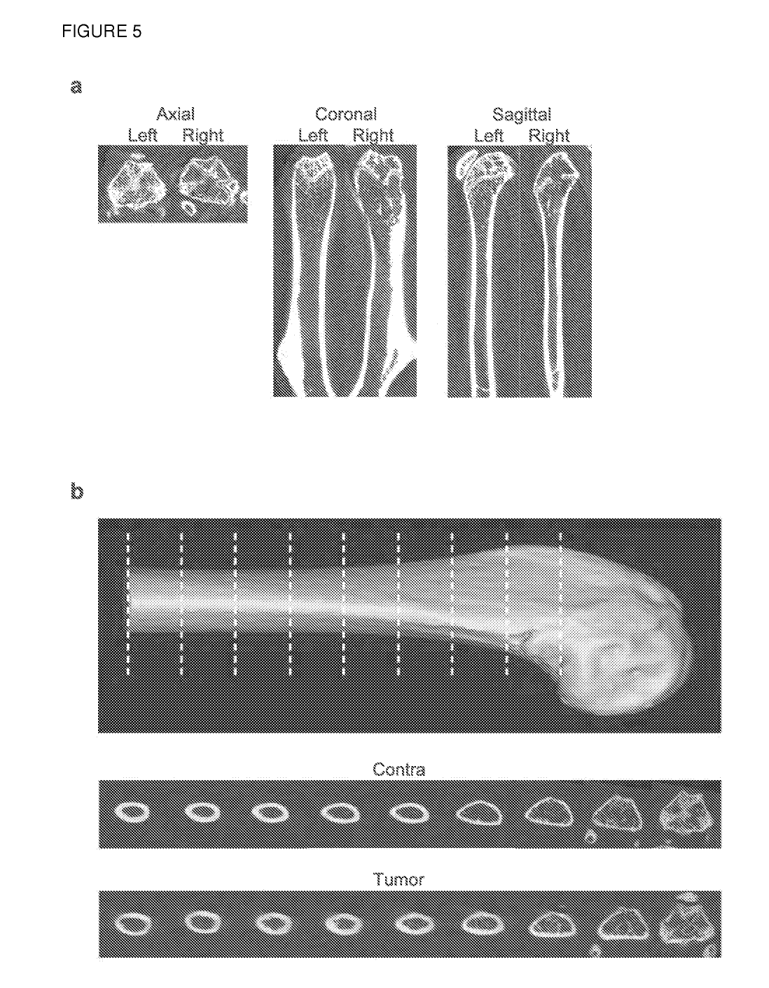

Micro computed tomography small animal scanning (microCT) was performed on mice injected intra-femorally with PCSD1 to determine the effect of the growth of the tumors [31]. MicroCT scans from mice injected intra-femorally with the primary patient bone metastasis sample are shown in FIG. 4. Regions with significant osteoporosis (bone thinning) as well as areas of bone sclerosis (increased bone density) were apparent in the femur in which the tumor was growing but not in the un-injected contra-lateral leg (FIG. 4A). Three-dimensional (3D) reconstruction of the microCT scans revealed the extensively pitted, porous and eroded femur head from the tumor-injected (yellow) leg compared to the smooth femur surface contours of the contra-lateral, uninjected leg (FIG. 4B). PCSD1 tumor growth produced significant osteolysis in the femur. In addition, regions of sclerosis or osteoblastic lesion formation were observed in the right, tumor injected femurs as shown in FIG. 5A. MicroCT cross-sections along the length of the femur were compared to show the increased thickness and density of the femur in which PCSD1 tumor was growing compared to the un-injected femur (FIG. 5B). Therefore, in vivo microCT scanning revealed PCSD1 tumor growth produced mixed osteolytic and osteoblastic lesions. This recapitulated the mixed osteoblastic and osteolytic bone lesions observed in the patient.

### Discussion

Prostate cancer progression is marked by metastasis to bone, resistance to androgen deprivation therapy, radiotherapy and chemotherapy as well as the emergence of an apoptosis-resistant, tumor-initiating population for which there is no effective therapy [34, 60-66]. There is a pressing need for new models to investigate prostate cancer interaction with the bone microenvironment and to develop therapies but they have been difficult to establish due to poor take-rates of xenograft transplantation of primary prostate tumors [17-19]. Described herein is a bone metastasis-derived prostate cancer intra-femoral xenograft model (the “PCSD model”) for studying prostate metastatic bone disease. PCSD1 generated serially-transplantable sub-cutaneous and intra-femoral tumors when transplanted into immunodeficient Rag2^{-/-};γ_{c}^{-/-} male mice. PCSD1 xenograft tumors were characterized as PSA⁺, AR⁺, K5⁻, K14⁻, K8⁺, K18⁺, AMACR⁺, NKX3.1⁺, and TMPRSS2:ERG⁻ human prostate cancer. These biomarkers identified PCSD1 as an advanced luminal prostate cancer bone metastatic cancer [43-47, 51, 63, 64]. MicroCT analyses revealed PCSD1 formed mixed osteoblastic and osteolytic lesions in a murine femoral injection model which closely resembled the bone lesions in the patient [28, 31, 60].

In alternative embodiments, the PCSD1 xenograft model is used to understand the development of prostate cancer in the bone microenvironment, e.g., a castrate-resistant prostate cancer in the bone microenvironment. Tumor growth of PCSD1 xenografts in intact versus surgically castrated mice can be measured. In culture, PCSD1 cells demonstrated androgen-independence as they can survive and proliferate without the addition on androgens.

Current standard-of-care therapies such as bisphosphonates, radiation, anti-androgens, chemotherapy, such as docetaxel, often eventually fail in patients who develop castrate-resistant prostate cancer [1, 5, 15, 67-70]. The PCSD1 model will be used not only to elucidate mechanisms of failure of standard-of-care therapy but also to develop new therapies alone or in combination with current therapies.

The PCSD1 model will also be used to gain understanding of the unexpected, discordant effects of some new prostate cancer therapies that are being reported for bone metastatic prostate cancer. For example, in a Phase II Study of the new anti-androgen, Abiraterone, it was found that approximately one third patients with chemotherapy-naive metastatic castration-resistant prostate cancer displayed bone scan flare discordant with PSA serologic response [71]. In other words, many patients with significantly lowered PSA levels after treatment with abiraterone still showed positive bone scans [71]. Conversely, some patients treated with the new c-Met tyrosine kinase inhibitor, Cabozantinib (c-Met TKI, XL184), showed dramatic reductions in positive bone scans but, paradoxically, no decrease in their PSA levels [72]. New bone metastasis models such as PCSD1 are, therefore, essential to understand the complex mechanisms of interaction of prostate cancer with the bone microenvironment and the variation in response to therapies in different patients, types of bone lesions or stages of bone metastatic prostate cancer progression.

### Conclusions

PCSD1 xenografts tumors were characterized as advanced, luminal epithelial prostate cancer from a bone metastasis using RT-PCR and immunohistochemical biomarker analyses. PCSD1 intra-femoral xenografts formed mixed osteoblastic/osteolytic lesions that closely resembled the bone lesions in the patient. PCSD1 is a new primary prostate cancer bone metastasis-derived xenograft model to study metastatic disease in the bone and to develop novel therapies for inhibiting prostate cancer growth in the bone-niche.

### Abbreviations

AR—androgen receptor, IHC—Immunohistochemistry, FACS—fluorescence activated cell scanning or sorting, IF—intra-femoral injection or transplantation, K562—Chronic myelogenous leukemia derived human cell line, LAPC4—Los Angeles Prostate Cancer cell line 4, LAPC9—Los Angeles Prostate Cancer cell line 9, Micro-CT—X-ray micro-computed tomography, P0—primagraft: primary patient sample injected, P1—first serial passage of tumor cells, that is, tumor cells harvested from P0 tumors are re-implanted into new mice and tumors allowed to develop, P2—second serial passage of xenograft tumors, PCSD1—Prostate Cancer San Diego 1 patient-derived xenograft or tumor cells, PSA—prostate specific antigen, Rag2^{-/-};γ_{c}^{-/-}—immunodeficient mouse strain with homozygous targeted deletions of Recombinase activated gene-2 and Interleukin 2 receptor common gamma chain, RT-PCR—Reverse transcription and polymerase chain reaction, SC—sub-cutaneous injection or transplantation, VCaP—vertebral metastasis of cancer of the prostate cell line.

TABLE 1
Primer sequences for RT-PCR analysis.

| Oligo Name* | Oligo Sequence (5′ -- 3′)* |
|---|---|
| h-PSA-F | ACCATGTGGGTCCCGGTTGT |
| h-PSA-R | GAGTTGATAGGGGTGCTCAGG |
| h-ARfl-F [48, 49] | ACATCAAGGAACTCGATCGTATCATTGC |
| h-ARfl-R [48, 49] | TTGGGCACTTGCACAGAGAT |
| h-ARv567es-F [48, 49] | CCAAGGCCTTGCCTGATTGC |
| h-ARv567es-R [48, 49] | TTGGGCACTTGCACAGAGAT |
| h-KRT5-F2 | CACCAAGACTGTGAGGCAGA |
| h-KRT5-R2 | CCTTGTTCATGTAGGCAGCA |
| h-KRT8-F [50] | CCTCATCAAGAAGGATGTGGA |
| h-KRT8-R [50] | CACCACAGATGTGTCCGAGA |
| h-KRT14-F [50] | GACCATTGAGGACCTGAGGA |
| h-KRT14-R [50] | ATTGATGTCGGCTTCCACAC |
| h-KRT18-F1 | CCAGTCTGTGGAGAACGACA |
| h-KRT18-R1 | CTGAGATTTGGGGGCATCTA |
| h-KRT18-F2 | CCAGTCTGTGGAGAACGACA |
| h-KRT18-R2 | ATCTGGGCTTGTAGGCCTTT |
| h-NKX3.1-F [46] | GGCCTGGGAGTCTTTGACTCCACTAC |
| h-NKX3.1-R [46] | ATGTGGAGCCCAAACCACAGAAAATG |
| h-AMACR-F [51] | CGCGGTGTCATGGAGAAACT |
| h-AMACR-R [51] | CTTCCTGACTGGCCAAATCC |
| h-GAPDH-F [52] | GGTGGTCTCCTCTGACTTCAACA |
| h-GAPDH-R [52] | TTGCTGTAGCCAAATTCGTTGT |
| m-Gapdh-F [53] | TGTTCCTACCCCCAATGTGT |
| m-Gapdh-R [53] | GGTCCTCAGTGTAGCCCAAG |
| TMPRSS2-ERG-F[54] | TAGGCGCGAGCTAAGCAGGAG |
| TMPRSS2-ERG-R[54] | GTAGGCACACTCAAACAACGACTGG |

TABLE 2
PCSD1 tumor xenograft passaging and transplantation take-rate.

| Tumor | | | | |
|---|---|---|---|---|
| Passage | | Number of | Number mice | |
| No. | | mice injected | with tumor | Take-rate |
| P0 | SC | 10 | 10 | 100% |
| | IF | 8 | 8 | 100% |
| P1 | SC | 18 | 18 | 100% |
| | IF | 29 | 19 | 66% |
| P2 | SC | 10 | 10 | 100% |
| | IF | 23 | 14 | 67% |

Tumor passage number: P0=primagraft: primary patient sample injected; P1=first serial passage of tumor cells, that is, tumor cells harvested from P0 tumors are re-implanted into new mice and tumors allowed to develop; P2=second serial passage of xenograft tumors; SC=sub-cutaneously transplanted tumors; IF=intra-femorally transplanted tumors.

### REFERENCES

1. Sturge J, Caley M P, Waxman J: Bone metastasis in prostate cancer: emerging therapeutic strategies. Nat Rev Clin Oncol 2011, 8:357-68.
2. Mehra R, Kumar-Sinha C, Shankar S, Lonigro R J, Jing X, Philips N E, Siddiqui J, Han B, Cao X, Smith D C, Shah R B, Chinnaiyan A M, Pienta K J: Characterization of bone metastases from rapid autopsies of prostate cancer patients. Clin Cancer Res. 2011, 17:3924-32.
3. Koeneman K S, Yeung F, Chung L W: Osteomimetic properties of prostate cancer cells: a hypothesis supporting the predilection of prostate cancer metastasis and growth in the bone environment. Prostate 1999, 39:246-261.
4. Virk M S, Lieberman J R: Tumor metastasis to bone. Arthritis Res Ther 2007, 9 (Suppl 1):S5.
5. Kim H S, Freedland S J: Androgen deprivation therapy in prostate cancer: anticipated side-effects and their management. Curr Opin Support Palliat Care 2010, 4:147-52.
6. Guise T: Examining the metastatic niche: targeting the microenvironment. Semin Oncol 2010, 37(Suppl 2):S2-14.
7. Suva L J, Washam C, Nicholas R W, Griffin R J: Bone metastasis: mechanisms and therapeutic opportunities. Nat Rev Endocrinol 2011, 7:208-18.
8. Wilson C, Coleman R E: Adjuvant therapy with bone-targeted agents. Curr Opin Support Palliat Care 2011, 5:241-50.
9. Body J J: New developments for treatment and prevention of bone metastases. Curr Opin Oncol 2011, 23:338-42.
10. Puhaindran M E, Farooki A, Steensma M R, Hameed M, Healey J H, Boland P J: Atypical subtrochanteric femoral fractures in patients with skeletal malignant involvement treated with intravenous bisphosphonates. J Bone Joint Surg Am 2011, 93:1235-42.
11. Henry D H, Costa L, Goldwasser F, Hirsh V, Hungria V, Prausova J, Scagliotti G V, Sleeboom H, Spencer A, Vadhan-Raj S, von Moos R, Willenbacher W, Woll P J, Wang J, Jiang Q, Jun S, Dansey R, Yeh H. J: Randomized, double-blind study of denosumab versus zoledronic acid in the treatment of bone metastases in patients with advanced cancer (excluding breast and prostate cancer) or multiple myeloma. Clin Oncol 2011, 29:1125-32.
12. Lee R J, Saylor P J, Smith M R: Treatment and prevention of bone complications from prostate cancer. Bone 2011, 48:88-95.
13. Fizazi K, Carducci M, Smith M, Damido R, Brown J, Karsh L, Milecki P, Shore N, Rader M, Wang H, Jiang Q, Tadros S, Dansey R, Goessl C: Denosumab versus zoledronic acid for treatment of bone metastases in men with castration-resistant prostate cancer: a randomised, double-blind study. Lancet 2011, 377:813-22.
14. Sartor O: Denosumab in bone-metastatic prostate cancer: known effects on skeletal-related events but unknown effects on quality of life. Asian J Androl 2011, 13:612-3.
15. Sonpavde G, Sternberg C N: Contemporary management of metastatic castration-resistant prostate cancer. Curr Opin Urol 2011, 21:241-7.
16. Smith, M. R., Brown, G. A., Saad, F: New opportunities in the management of prostate cancer-related bone complications. Urologic Oncology: Seminars and Original Investigations 2009, 27(Suppl. 1): S1-S20.
17. Hurwitz A A, Foster B A, Allison J P, Greenberg N M, Kwon E D: The TRAMP mouse as a model for prostate cancer. 2001, Current Protocols in Immunology 20.5.1-20.5.23.
18. Pienta K J, Abate-Shen C, Agus D B, Attar R M, Chung L W, Greenberg N M, Hahn W C, Isaacs J T, Navone N M, Peehl D M, Simons J W, Solit D B, Soule H R, VanDyke T A, Weber M J, Wu L, Vessella R L: The current state of preclinical prostate cancer animal models. Prostate 2008, 68:629-39.
19. Lopez-Barcons L A: Human prostate cancer heterotransplants: a review on this experimental model. Asian J Androl 2010, 12:509-18.
20. Corey E, Quinn J E, Bladou F, Brown L G, Roudier M P, Brown J M, Buhler K R, Vessella R L: Establishment and characterization of osseous prostate cancer models: intra-tibial injection of human prostate cancer cells. Prostate 2002, 52:20-33.
21. Davies M R, Lee Y P, Lee C, Zhang X, Afar D E, Lieberman J R: Use of a SCID mouse model to select for a more aggressive strain of prostate cancer. Anticancer Res 2003, 23:2245-52.
22. Lin D L, Tarnowski C P, Zhang J, Dai J, Rohn E, Patel A H, Morris M D, Keller E T: Bone metastatic LNCaP-derivative C4-2B prostate cancer cell line mineralizes in vitro. Prostate 2001, 47:212-221.
23. Morrissey C, Kostenuik P L, Brown L G, Vessella R L, Corey E: Host-derived RANKL is responsible for osteolysis in a C4-2 human prostate cancer xenograft model of experimental bone metastases. BMC Cancer 2007, 7:148.
24. Tsingotjidou A S, Zotalis G, et al: Development of an animal model for prostate cancer cell metastasis to adult human bone. Anticancer Res 2001, 21:971-8.
25. Lee Y, Schwarz E, Davies M, Jo M, Gates J, Wu J, Zhang X, Lieberman J R: Differences in the cytokine profiles associated with prostate cancer cell induced osteoblastic and osteolytic lesions in bone. J Orthop Res 2003, 21:62-72.
26. Craft, N., Chor, C., et al: Evidence for clonal outgrowth of androgen-independent prostate cancer cells from androgen-dependent tumors through a two-step process. Cancer Research 1999, 59:5030-5036.
27. Gamradt S C, Feeley B T, Liu N Q, Roostaeian J, Lin Y Q, Zhu L X, Sharma S, Dubinett S M, Lieberman J R: The effect of cyclooxygenase-2 (COX-2) inhibition on human prostate cancer induced osteoblastic and osteolytic lesions in bone. Anticancer Res 2005, 25:107-15.
28. Feeley, B., Gamradt, S C, Hsu, W K et al: Influence of BMPs on the formation of osteoblastic lesions in metastatic prostate cancer. J Bone and Mineral Research 2005, 20:2189-2199.
29. Ristevski B, Jenkinson R J, Stephen D J, Finkelstein J, Schemitsch E H, McKee M D, Kreder H J: Mortality and complications following stabilization of femoral metastatic lesions: a population-based study of regional variation and outcome. Can J Surg 2009, 52:302-308.
30. Piccioli A, Maccauro G, Rossi B, Scaramuzzo L, Frenos F, Capanna R: Surgical treatment of pathologic fractures of humerus. Injury 2010, 41:1112-6.
31. Hsu W K, Virk M S, Feeley B T, Stout D B, Chatziioannou A F, Lieberman J R: Characterization of osteolytic, osteoblastic, and mixed lesions in a prostate cancer mouse model using 18F-FDG and 18F-fluoride PET/CT. J Nucl Med 2008 March; 49(3):414-21. Epub 2008 Feb. 20.
32. Li X, Loberg R, Liao J, Ying C, Snyder L A, Pienta K J, McCauley L K: A destructive cascade mediated by CCL2 facilitates prostate cancer growth in bone. Cancer Res 2009, 69:1685-92.
33. Klein, K. A., Reiter, R. E., Redula, J., Moradi, H., Zhu, X. L., Brothman, A. R., Lamb, D. J., Marcelli, M., Belldegrun, A., Witte, O. N. and Sawyers, C. L: Progression of metastatic human prostate cancer to androgen independence in immunodeficient SCID mice. Nat. Med 1997, 3:402-408.
34. Nickerson T, Chang F, Lorimer D et al: In vivo progression of LAPC-9 and LNCaP prostate cancer models to androgen independence is associated with increased expression of insulin-like growth factor I (IGF-I) and IGF-I receptor (IGF-IR): Cancer Res 2001, 61:6276-80.
35. Sandberg R, Ernberg I: The molecular portrait of in vitro growth by meta-analysis of gene-expression profiles. Genome Biol 2005, 6: R65.
36. Taylor B S, Schultz N, Hieronymus H, Gopalan A, Xiao Y, Carver B S, Arora V K, Kaushik P, Cerami E, Reva B, Antipin Y, Mitsiades N, Landers T, Dolgalev I, Major J E, Wilson M, Socci N D, Lash A E, Heguy A, Eastham J A, Scher H I, Reuter V E, Scardino P T, Sander C, Sawyers C L, Gerald W L: Integrative genomic profiling of human prostate cancer. Cancer Cell 2010, 18:11-22.
37. Berger M F, Lawrence M S, Demichelis F, Drier Y, Cibulskis K, Sivachenko A Y, Sboner A, Esgueva R, Pflueger D, Sougnez C, Onofrio R, Carter S L, Park K, Habegger L, Ambrogio L, Fennell T, Parkin M, Saksena G, Voet D, Ramos A H, Pugh T J, Wilkinson J, Fisher S, Winckler W, Mahan S, Ardlie K, Baldwin J, Simons J W, Kitabayashi N, MacDonald T Y, Kantoff P W, Chin L, Gabriel S B, Gerstein M B, Golub T R, Meyerson M, Tewari A, Lander E S, Getz G, Rubin M A, Garraway L A: The genomic complexity of primary human prostate cancer. Nature 2011, 470: 214-20.
38. Kim J H, Dhanasekaran S M, Prensner J R, Cao X, Robinson D, Kalyana-Sundaram S, Huang C, Shankar S, Jing X, Iyer M, Hu M, Sam L, Grasso C, Maher C A, Palanisamy N, Mehra R, Kominsky H D, Siddiqui J, Yu J, Qin Z S, Chinnaiyan A M: Deep sequencing reveals distinct patterns of DNA methylation in prostate cancer. Genome Res 2011, 21:1028-41.
39. Virk M S, Petrigliano F A, Liu N Q, Chatziioannou A F, Stout D, Kang C O, Dougall W C, Lieberman J R: Influence of simultaneous targeting of the bone morphogenetic protein pathway and RANK/RANKL axis in osteolytic prostate cancer lesion in bone: Bone 2009, 44:160-7.
40. Hung T T, Chan J, Russell P J, Power C A: Zoledronic acid preserves bone structure and increases survival but does not limit tumour incidence in a prostate cancer bone metastasis model. PLoS One 2011, 6: e19389.
41. Bruni-Cardoso A, Johnson L C, Vessella R L, Peterson T E, Lynch C C: Osteoclast-Derived Matrix Metalloproteinase-9 Directly Affects Angiogenesis in the Prostate Tumor-Bone Microenvironment. Mol Cancer Res 2010, 8: 459-70.
42. Chen C D, Welsbie D S, Tran C, et al: Molecular determinants of resistance to antiandrogen therapy. Nat Med 2004, 10: 33-39.
43. Collins A T, Berry P A, Hyde C, et al: Prospective identification of tumorigenic prostate cancer stem cells. Cancer Res 2005, 65: 10946-10951.
44. Duhagon M A, Hurte E M, Sotelo-Silveira J R, Zhang X, Farrar W L: Genomic profiling of tumor initiating prostatospheres. BMC Genomics 2010, 11: 324.
45. Garraway I P, Sun W, Tran C P, et al: Human prostate sphere-forming cells represent a subset of basal epithelial cells capable of glandular regeneration in vivo. Prostate 2010, 70:491-501.
46. Goldstein A S, Huang J, Guo C, Garraway I P, Witte O N: Identification of a cell of origin for human prostate cancer. Science 2010, 329:568-71.
47. Gregg J L, Brown K E, Mintz E M, et al: Analysis of gene expression in prostate cancer epithelial and interstitial stromal cells using laser capture microdissection. BMC Cancer 2010, 10:165.
48. Sun S, Sprenger C C, Vessella R L, Haugk K, Soriano K, Mostaghel E A, Page S T, Coleman I M, Nguyen H M, Sun H, Nelson P S, Plymate S R: Castration resistance in human prostate cancer is conferred by a frequently occurring androgen receptor splice variant. J Clin Invest 2010, 120: 2715-30.
49. Watson P A, Chen Y F, Balbas M D, Wongvipat J, Socci N D, Viale A, Kim K, Sawyers C L: Constitutively active androgen receptor splice variants expressed in castration-resistant prostate cancer require full-length androgen receptor. Proc Natl Acad Sci USA 2010, 107:16759-65.
50. Lawson D A, Zong Y, Memarzadeh S, Xin L, et al: Basal epithelial stem cells are efficient targets for prostate cancer initiation. Proc Natl Acad Sci USA 2010, 107: 2610-5.
51. Ouyang B, Leung Y K, Wang V, Chung E, Levin L, Bracken B, Cheng L, Ho S M: α-Methylacyl-CoA racemase spliced variants and their expression in normal and malignant prostate tissues. Urology 2011, 77: 249.e1-7.
52. Johnson M B, Kawasawa Y I, Mason C E, et al: Functional and evolutionary insights into human brain development through global transcriptome analysis. Neuron 2009, 62: 494-509.
53. Nervina J M, Magyar C E, Pirih F Q, Tetradis S: PGC-1alpha is induced by parathyroid hormone and coactivates Nurrl-mediated promoter activity in osteoblasts. Bone 2006, 39: 1018-25.
54. Tomlins S A, Rhodes D R, Perner S, Dhanasekaran S M, Mehra R, Sun X W, Varambally S, Cao X, Tchinda J, Kuefer R, Lee C, Montie J E, Shah R B, Pienta K J, Rubin M A, Chinnaiyan A M: Recurrent fusion of TMPRSS2 and ETS transcription factor genes in prostate cancer. Science 2005, 310: 644-8.
55. Mitra N, Banda K, Altheide T K, Schaffer L, Johnson-Pais T L, Beuten J, Leach R J, Angata T, Varki N, Varki A: SIGLEC12, a Human-specific Segregating (Pseudo)gene, Encodes a Signaling Molecule Expressed in Prostate Carcinomas. J Biol Chem 2011, 286: 23003-11.
56. Lavoie et al: Skin-derived precursors differentiate into skeletogenic cell types and contribute to bone repair. Stem Cells and Development 2009, 18: 893-906.
57. Ritacco, L. E., et al: Three-dimensional morphometric analysis of the distal femur: a validity method for allograft selection using a virtual bone bank. Stud Health Technol Inform 2010, 160: p. 1287-90.
58. Ochia, R. S., et al.: Three-dimensional in vivo measurement of lumbar spine segmental motion. Spine 2006, 31: 2073-8.
59. Abrahamsson A E, Geron I, Gotlib J, Dao K H, Barroga C F, Newton I G, Giles F J, Durocher J, Creusot R S, Karimi M, Jones C, Zehnder J L, Keating A, Negrin R S, Weissman I L, Jamieson C H: Glycogen synthase kinase 3beta missplicing contributes to leukemia stem cell generation. Proc Natl Acad Sci USA 2009, 106: 3925-9.
60. Ibrahim T, Flamini E, Mercatali L, Sacanna E, Sena P, Amadori D: Pathogenesis of osteoblastic bone metastases from prostate cancer. Cancer 2010, 116:1406-18.
61. Grubb R L, Deng J, Pinto P A et al. Pathway biomarker profiling of localized and metastatic human prostate cancer reveal metastatic and prognostic signatures. J Proteome Res. 2009, 8:3044-54.
62. Ugolkov A V, Eisengart L J, Luan C, Yang X J: Expression analysis of putative stem cell markers in human benign and malignant prostate. Prostate 2010, Jun. 25. [Epub ahead of print]
63. Wang X, Kruithof-de Julio M, Economides K D, et al: A luminal epithelial stem cell that is a cell of origin for prostate cancer. Nature 2009, 461: 495-500.
64. Petrigliano F A, Virk M S, Liu N, Sugiyama O, Yu D, Lieberman J R: Targeting of prostate cancer cells by a cytotoxic lentiviral vector containing a prostate stem cell antigen (PSCA) promoter. Prostate 2009, 69:1422-34.
65. Schayek H, Seti H, Greenberg N M, Sun S, Werner H, Plymate S R: Differential regulation of insulin-like growth factor-I receptor gene expression by wild type and mutant androgen receptor in prostate cancer cells. Mol Cell Endocrinol 2010, 323: 239-45.
66. Klarmann G J, Hurt E M, Mathews L A, Zhang X, Duhagon M A, Mistree T, Thomas S B, Farrar W L: Invasive prostate cancer cells are tumor initiating cells that have a stem cell-like genomic signature. Clin Exp Metastasis 2009, 26:433-46.
67. Koreckij T, Nguyen H, Brown L G, Yu E Y, Vessella R L, Corey E: Dasatinib inhibits the growth of prostate cancer in bone and provides additional protection from osteolysis. Br J Cancer 2009, 101:263-8.
68. Tang Y, Hamburger A W, Wang L, et al: Androgen deprivation and stem cell markers in prostate cancers. Int J Clin Exp Pathol 2009, 3:128-38.
69. Tran C, Ouk S, Clegg N J, Chen Y, Watson P A, Arora V, Wongvipat J, Smith-Jones P M, Yoo D, Kwon A, Wasielewska T, Welsbie D, Chen C D, Higano C S, Beer T M, Hung D T, Scher H I, Jung M E, Sawyers C L: Development of a second-generation antiandrogen for treatment of advanced prostate cancer. Science 2009, 324:787-90.
70. Scher H I, Beer T M, Higano C S et al: Antitumour activity of MDV3100 in castration-resistant prostate cancer: a phase 1-2 study. Lancet 2010, 375:1437-46.
71. Ryan C J, Shah S, Efstathiou E, Smith M R, Taplin M E, Bubley G J, Logothetis C J, Kheoh T, Kilian C, Haqq C M, Molina A, Small E J: Phase II study of abiraterone acetate in chemotherapy-naive metastatic castration-resistant prostate cancer displaying bone flare discordant with serologic response. Clin Cancer Res 2011, 17:4854-61.
72. Scholz, M: Cabozantinib PCRI Insights 2011, 14: 12-14.

A number of embodiments have been described herein. Nevertheless, it will be understood that various modifications may be made without departing from the spirit and scope of the embodiments disclosed. Accordingly, other embodiments are within the scope of the following claims.

1. A method for making a bone metastasis-derived prostate cancer xenograft model, comprising:
(a) providing an immunodeficient non-human animal, or a non-human animal lacking B, T and NK cells,
wherein optionally the non-human animal is:
a murine animal, or an animal of the subfamily Murinae or in the family Muridae,
a rat or a mouse,
an immunodeficient rat or mouse or animal of the subfamily Murinae or in the family Muridae, or a male rat or mouse or animal of the subfamily Murinae or in the family Muridae,
an immunodeficient Rag2^{-/-};γ_{c}^{-/-} male rat or mouse or animal of the subfamily Murinae or in the family Muridae,
a male non-human animal strain equivalent having the equivalent of a Rag2^{-/-};γ_{c}^{-/-} genotype and/or phenotype;
an immunodeficient mouse or animal of the subfamily Murinae or in the family Muridae comprising (having contained therein) a human growth factor transgene or transgenes, wherein optionally the immunodeficient mouse is a Jackson Laboratory NOD.Cg-Prkdc^{scidIl2rgtm1Wjl}/SzJ; or
an immunodeficient murine or mouse strain or animal of the subfamily Murinae or in the family Muridae having a complete or partial human immune system reconstitution and/or modification;
(b) providing a mammalian or a human prostate cancer cell or cells,
wherein optionally the cell or cells are not passaged or cultured, or optionally the cell or cells are cultured for one, two, three, four or five or more passages,
and optionally unpassaged or uncultured cells, or the optionally passaged or cultured cells, are cryopreserved, lyophilized, freeze-dried or otherwise stored before placement (insertion) into the non-human animal,
and optionally the cells or cells are initially derived from a biopsy; and
(c) placing, injecting or inserting the human prostate cancer cell or cells into the non-human animal;
wherein optionally the cell or cells are injected, placed or inserted by injection subcutaneously (SQ), intravenously (IV), or directly into bone, or intra-femorally (IF), and/or intradermally.

2. The method of any of claim 1, wherein the mammalian or human prostate cancer cell or cells are an advanced luminal prostate cancer bone metastatic cancer cell or cells, or have a human prostate cancer phenotype or genotype comprising: PSA⁺, AR⁺, K5⁻, K14⁻, K8⁺, K18⁺, AMACR⁻, NKX3.1⁺, and TMPRSS2:ERG⁻.

3. The method of claim 1, wherein the mammalian or human cell or cells, or the biopsy, is or are derived from a mixed osteoblastic and/or an osteolytic lesion.

4. A bone metastasis-derived prostate cancer xenograft non-human animal or animal model made by the method comprising or consisting of the method of claim 1.

5. A method for testing whether a drug, compound, diet, therapy or treatment is effective or efficacious for: preventing, ameliorating, slowing the progress of, stopping or slowing the metastasis of, or for causing a full or partial remission of a cancer, or a prostate cancer, or a human prostate cancer; or, modifies or effects the structure or organization or vascularization of a tumor microenvironment; or effects the growth, survival, phenotype or histology (tissue or organ structure or microenvironments) of connective tissue, bone cells, osteoblasts, osteocytes, osteoclasts, bone marrow cells, fibroblasts or angiogenic cells, comprising:
administering or applying the drug, compound, diet, therapy or treatment to the bone metastasis-derived prostate cancer xenograft model of claim 1, and
after administering or applying the drug, compound, diet, therapy or treatment to the bone metastasis-derived prostate cancer xenograft model, determining or measuring the effect of the drug, compound, diet, therapy or treatment on the cancer cells, or the effect of the drug, compound, diet, therapy or treatment on cells or composition or structure or organization or vascularization of a tumor microenvironment, or on connective tissue, bone cells, osteoblasts, osteocytes, osteoclasts, bone marrow cells, fibroblasts or angiogenic cells,
wherein optionally it is measured determined whether the drug, compound, diet, therapy or treatment is effective or efficacious for preventing, ameliorating, slowing the progress of, stopping or slowing the metastasis of, or for causing a full or partial remission of, a cancer, or a prostate cancer, or a human prostate cancer,
and optionally it is measured determined whether the drug, compound, diet, therapy or treatment is effective or efficacious for preventing, ameliorating, slowing the progress of, stopping or slowing a bone lesion formation, or effects bone or effects osteolysis, osteosclerosis, osteoporosis, or osteopetrosis caused by a cancer, a prostate cancer or a human prostate cancer or cells,
and optionally a drug, compound, diet, therapy or treatment known to be effective or efficacious is co-administered, or administered to the same or similar animal, as a positive control.

6. An immunocompromised mouse model in which PCSD1 cells are transplanted subcutaneously (SQ), intravenously (IV), or directly into bone, or intra-femorally (IF), and/or intradermally and a cancer forms.

7. The mouse model of claim 6, wherein the PCSD1 cells are transplanted intrafemorally and the cancer is bone cancer.

8. The mouse model of claim 7, wherein the bone cancer has osteolytic and osteoblastic lesions.

9. The mouse model of claim 6, wherein the cancer expresses AR, NKX3.1, Keratins 8 and 18 and AMACR.

10. The mouse model of claim 6, wherein the immunocompromised mouse is NOD.Cg-Prkdc^{scidIl2rgtm1Wjl}/SzJ.

11. A screening method of therapeutic agents for prostate cancer that has metastasized to bone, which comprises administering a test substance to the PCSD-1 mouse model of claim 6.

12. A bone metastasis-derived prostate cancer xenograft non-human animal or animal model made by the method comprising or consisting of the method of claim 2

13. A bone metastasis-derived prostate cancer xenograft non-human animal or animal model made by the method comprising or consisting of the method of claim 3.

14. The method of claim 5, wherein the mammalian or human prostate cancer cell or cells are an advanced luminal prostate cancer bone metastatic cancer cell or cells, or have a human prostate cancer phenotype or genotype comprising: PSA⁺, AR⁺, K5⁻, K14⁻, K8⁺, K18⁺, AMACR⁺, NKX3.1⁺, and TMPRSS2:ERG⁻.

15. The method of claim 5, wherein the mammalian or human cell or cells, or the biopsy, is or are derived from a mixed osteoblastic and/or an osteolytic lesion.
